(19) United States
(12) Patent Application Publication
(10) Pub. No.: US 2024/0384217 A1
(43) Pub. Date: Nov. 21, 2024

(54) METHOD AND DEVICE FOR MONITORING MICROBIAL GROWTH, USE OF A PACKAGED NUTRIENT MEDIUM

(71) Applicant: SKAN AG, Allschwil (CH)

(72) Inventors: Christoph Joschi Geiser, Bottmingen (CH); Mathieu MÜller, Sierentz (FR); Marc Tondera, Riehen (CH); Florian Micha Weiss, ZÜrich (CH); Mike Zeller, Wahlen (CH)

(73) Assignee: SKAN AG, Allschwil (CH)

(21) Appl. No.: 18/656,978

(22) Filed: May 7, 2024

(30) Foreign Application Priority Data
May 17, 2023 (DE) 102023113119.2

(51) Int. Cl. C12M 1/12; C12M 1/34

(52) U.S. Cl. CPC C12M 37/06; C12M 37/00; C12M 41/00

(57) ABSTRACT

For monitoring for microbial growth, it is provided that a fluid flow is discharged via a fluid line from a cleanroom into a controlled environment, wherein a nutrient medium is presented in the controlled environment and with which the fluid flow is contacted and subsequently removed from the controlled environment in such a way that a cleanroom process does not have to be interrupted.

### CROSS-REFERENCE TO RELATED APPLICATIONS

This application claims priority from German Patent Application No. 10 2023 113 119.2, filed May 17, 2023, which is incorporated herein by reference as if fully set forth.

### TECHNICAL FIELD

The invention relates to a method for monitoring for microbiological contamination in a cleanroom, preferably an isolator, wherein a fluid flow, in particular an air flow, is discharged from the cleanroom.

The invention also relates to a device for monitoring for microbiological contamination in a cleanroom.

Finally, the invention relates to a use, in particular within a cleanroom and/or within a controlled environment, of a packaged nutrient medium, in particular impactor, for presenting the nutrient medium in a fluid flow, wherein the fluid flow has been taken from a withdrawal location from a cleanroom, in particular isolator, in particular wherein the impactor has a nozzle plate behind which the nutrient medium is arranged, wherein the nutrient medium, in particular behind the nozzle plate, is covered by a closure before use.

### BACKGROUND

It is common practice to monitor a cleanroom for microbiological contamination by taking samples.

### SUMMARY

The invention is based on the object of monitoring for microbiological contamination within a cleanroom almost continuously, so that a cleanroom process does not have to be interrupted or only for a short time.

To solve this object, the invention proposes one or more of the features disclosed herein. In particular, it is thus proposed according to the invention in a method for monitoring for microbiological contamination that the fluid flow contacts a nutrient medium in a controlled environment arranged outside the cleanroom, preferably in an environment connected by a fluid line and/or separable from the cleanroom, wherein the nutrient medium is used for monitoring for microbiological contamination.

It may be provided in this case, for example, that the controlled environment can be separated from the cleanroom except for a supply line, for example the fluid line. It is particularly advantageous that a removal point from which the fluid flow can be discharged from the cleanroom, preferably the isolator, can be defined relatively freely.

It is also particularly advantageous that products in the cleanroom can be prevented from being contaminated, in particular by directly intervening in a cleanroom process taking place in the cleanroom. This means that the nutrient medium can be exchanged virtually indefinitely without an active airlock and without the cleanroom subsequently being decontaminated, for example by closing off the controlled environment from the cleanroom during the exchange and establishing the fluidic connection for a measurement after the exchange. A structurally simple design can be an arrangement of the controlled environment outside the cleanroom. This can improve accessibility for the exchange.

A cleanroom process can be, for example, aseptic filling and/or sealing and/or analysis of pharmaceutical products.

The nutrient medium can, for example, be solid, in particular as agar, or liquid, in particular as broth.

The fluid flow can be discharged from the cleanroom horizontally and/or vertically or at an angle, for example.

In general, in an advantageous application of the invention, the cleanroom can be designed as an isolator, for example.

It is particularly advantageous that various decontamination processes are minimized by contacting the fluid flow discharged from the cleanroom with the nutrient medium in a controlled environment.

In addition, it is particularly advantageous that the risk of contamination of the products manufactured in the cleanroom during a cleanroom process can also be minimized.

As monitoring for microbiological contamination takes place outside the cleanroom, it is particularly advantageous that this enables almost continuous or recurring measurement without the cleanroom process having to be interrupted for any length of time. This also makes it particularly advantageous to generate faster and more cost-effective process sequences that can be realized with fewer rejects.

Furthermore, it may be provided, for example, that at least one characteristic variable, in particular a volume flow of the fluid flow, is detected and/or that the characteristic variable is regulated, in particular on the basis of the detection.

In a further advantageous embodiment, it may be provided that an interface delimiting the controlled environment is decontaminated, in particular before the interface is made accessible to the fluid flow to be monitored.

The controlled environment limiting interface can be, for example, the nutrient medium that can be coupled and/or exchanged via a docking opening or an access opening.

A particular advantage is that this ensures that any detectable microbiological impurities can be clearly assigned to the fluid flow discharged from the cleanroom and are not caused by contamination of the interface.

This has the advantage, for example, of preventing organisms capable of multiplying at the interface, which have arisen due to contamination outside the cleanroom, resulting in false positive findings and unnecessary rejection of the products manufactured during the cleanroom process.

In a further advantageous embodiment, it may be provided that a branch or a diverter is formed on the fluid line, in particular wherein the fluid flow is controllable.

It is particularly advantageous that, for example, two separate devices can be used to monitor for microbiological contamination, which in turn enable continuous monitoring for microbiological contamination in the cleanroom. These can be operated overlapping or alternating in time.

For example, it may be provided that a nutrient medium can be presented in one of the two devices and contacted by the fluid flow discharged from the cleanroom via the fluid line. As soon as the nutrient medium is to be placed in an incubator, for example, and thus exchanged, a nutrient medium can be placed in the other device and the fluid line or the fluid flow can be diverted accordingly. In this way, the removal of one nutrient medium can advantageously take place simultaneously with continuous monitoring for microbiological contamination of the fluid flow from the cleanroom via the other nutrient medium.

In a further advantageous embodiment, it may be provided that a decontamination agent, in particular a gas or aerosol, preferably hydrogen peroxide, is supplied for decontaminating the interface and/or the controlled environment, in particular wherein the gas or aerosol is supplied from the cleanroom and/or from a separate decontamination source.

It is particularly advantageous in this case that the gas or aerosol used for decontamination can thus be supplied at different points of the interface and the usual loop processes for fumigation can be used.

For example, it may be provided in this case that the gas or aerosol may flow into the cleanroom, wherein the decontamination of the interface is carried out, for example, via the fluid flow discharged from the cleanroom.

In a further advantageous embodiment, it may be provided that the fluid flow is discharged from the cleanroom at a process point within the cleanroom, at which a cleanroom process is preferably carried out, and/or wherein the fluid flow is supplied to the nutrient medium in a fluid line which runs at least partially within the cleanroom. It is particularly advantageous that a continuous measurement, for example of the cleanroom air, is already possible during the cleanroom process via a direct line of the fluid flow, which means that the cleanroom process does not have to be interrupted or only for a short time.

In a further advantageous embodiment, it may be provided that the fluid flow is passed through a particle counter for detecting a particle count and/or particle size, in particular wherein the same fluid flow is subsequently passed to the nutrient medium and/or wherein a partial volume of the fluid flow is branched off, in particular wherein the branched-off fluid flow is transferred to a particle counter for detecting a particle count and/or wherein the branching-off takes place outside the cleanroom.

The fluid flow can be diverted in a controlled manner via adjustable and/or controllable nozzles, for example.

For example, it may be provided in this case that the particle counter can be arranged just before the controlled environment on a fluid line. It may also be possible, for example, for the particle counter to be located outside the controlled environment inside the cleanroom on a fluid line.

It is particularly advantageous in this case that the monitoring for impurities can be supplemented with quantitative information, such as the number and/or size of the impurities, using functionally different methods. A particle counter can also be used to narrow down the time of contamination more precisely.

Another advantage is that the particle counter can be used to determine the number of possible impurities, while the nutrient medium can be used to detect which type of impurity is present.

The advantage of such a combination is that it enables a faster response time for interrupting the cleanroom process.

In a further advantageous embodiment, it may be provided that the nutrient medium is introduced into the controlled environment from the outside and positioned at a receptacle, in particular wherein the controlled environment is decontaminated after introduction.

This makes it possible, for example, to replace the nutrient medium during a cleanroom process.

It is particularly advantageous in this case that the nutrient medium can thus be changed or exchanged during a cleanroom process and the cleanroom process can therefore be continued independently of the introduction of the nutrient medium without risk of contamination of the cleanroom. Accordingly, it is also advantageous that the cleanroom process can be continued if the controlled environment is contaminated.

In an advantageous embodiment, it may be provided that a backflow prevention device, in particular a valve, is preferably activated between the controlled environment and the cleanroom before an exchange of the nutrient medium.

For example, it may be provided that a valve needs to be used that offers a minimal contamination surface in order to prevent the probability of contamination of surfaces that are difficult to decontaminate.

It is particularly advantageous in this case that any possible contamination of the controlled environment during the exchange of the nutrient medium does not lead to contamination of the fluid line due to a backflow of the contaminated fluid flow.

In a further advantageous embodiment, it may be provided that after insertion, a closure of the nutrient medium is removed and stored in the controlled environment, in particular wherein the closure is stored outside a flow path of the fluid flow.

For example, it may be provided that the nutrient medium can be pressed against an outlet opening, an outlet chamber or an outlet cone of the fluid flow.

It is particularly advantageous that the removed closure does not negatively influence the flow properties of the fluid flow, which may result in a possibly arising turbulent flow of the fluid flow, for example. In addition, this advantageously prevents possible impurities adhering to the closure from being entrained by the fluid flow and distorting the measurement result or microorganisms brought in by the fluid flow adhering to the closure or a closure receptacle and thus not being transferred to the nutrient medium.

In particular, the closure receptacle is designed to surround the outside of a closure. If the outside of the closure is completely or practically completely covered by the closure receptacle, contamination from the outside can be ruled out (or practically ruled out). For example, this can be realized in the manner of a rapid transfer port.

Alternatively or additionally, one or more of the features noted below directed to a method are provided according to the invention for solving the said object. Thus, in particular, in order to solve the said object in a method for monitoring for microbiological contamination in a cleanroom, wherein a fluid flow, in particular an air flow, is discharged from the cleanroom, wherein the fluid flow contacts a nutrient medium, wherein the nutrient medium is used for monitoring for microbiological contamination, in particular as described above, it is proposed that a fluid line, which brings the fluid flow to the nutrient medium, is connected to the nutrient medium after a closure of the nutrient medium has been removed.

For example, it may be provided that the fluid line can be connected to the nutrient medium by mechanically connecting and/or opening a shut-off valve.

It is particularly advantageous in this case that a controlled room can be formed by the cleanroom and thus a controlled room arranged separately from the cleanroom can be dispensed with.

It is also advantageous that this prevents a potentially contaminated fluid flow from flowing back, as the fluid line is only connected to the nutrient medium when the nutrient medium is sealed.

In a further advantageous embodiment, it may be provided that the closure is removed via a transfer movement, in particular a preferably horizontal pivoting movement and/or a lifting movement.

It is particularly advantageous that the closure can thus be easily moved to an area outside the flow path of the fluid flow and simultaneously removed from the nutrient medium.

In a further advantageous embodiment, it may be provided that the nutrient medium, in particular on a nutrient medium carrier, is preferably moved vertically by a lifting device, in particular which is connectable or connected to a membrane, for removing and/or attaching the closure and/or for coupling the fluid line.

The nutrient medium carrier can, for example, form a housing for the nutrient medium.

For example, it may be provided in this case that the membrane can be designed to separate a space into which the closure is pivoted from an external space.

It is particularly advantageous in this case that decontamination can take place in this external space.

In a further advantageous embodiment, it may be provided that the nutrient medium is removed from the controlled environment after contact with the fluid flow, in particular by a movement of the receptacle.

For example, it may be provided in this case that the receptacle can be moved horizontally and/or vertically.

For example, it may also be provided that the nutrient medium is sealed with the closure after contact with the fluid flow before the nutrient medium is removed from the controlled environment.

It is particularly advantageous in this case that contamination of the controlled space can be prevented in this way as far as possible.

The advantage of this is that the nutrient medium can be easily removed and fed into an incubator, for example.

In a further advantageous embodiment, it may be provided that a particle detection is carried out in the fluid flow or a further fluid flow and one or the nutrient medium is only and/or automatically presented in the fluid flow when the particle detection has detected a preferably reproducible and/or living object, in particular by diverting the fluid flow, opening a fluid line conducting the fluid flow and/or starting the fluid flow.

It is particularly advantageous that the nutrient medium is thus only opened, used and/or consumed when the particle counter has been able to detect microbiological contamination.

This is another advantageous way of preventing the nutrient medium from drying out unnecessarily.

Alternatively or additionally, the features of the alternative independent claim directed to a device are provided according to the invention for solving the said object. In particular, it is thus proposed according to the invention, for solving the said object of monitoring for microbiological contamination in a cleanroom, that an enclosure which encloses a controlled environment, for example the aforementioned controlled environment, comprises a receptacle, for example the aforementioned receptacle, for presenting a nutrient medium, for example the aforementioned nutrient medium, in the controlled environment, a fluidic connection for connection to the cleanroom and an interface which delimits the controlled environment, in particular for providing the nutrient medium.

It is particularly advantageous that the advantages of the method already claimed can thus be realized.

In an advantageous embodiment, it may be provided that the fluidic connection has a branch or a diverter designed to divert a fluid flow.

It is particularly advantageous that a fluid flow, which is discharged in particular from the cleanroom, can be continuously monitored for microbiological contamination, for example by using two separate controlled environments in which a nutrient medium can be presented.

In a further advantageous embodiment, it may be provided that a decontamination device is formed, by means of which at least the interface, in particular the controlled environment, can be decontaminated.

For example, it may be provided in this case that the decontamination device is designed to supply a decontamination agent, in particular gas or aerosol, such as hydrogen peroxide, for example from the cleanroom and/or from a separate decontamination source to the interface in order to decontaminate the interface.

It is particularly advantageous in this case that it is therefore virtually impossible for microbiological contamination to be caused by a contaminated interface. This in turn has the advantage that any microbiological impurities detected are highly likely to have been caused by contamination of the cleanroom.

In a further advantageous embodiment, it may be provided that the interface is designed as part of the enclosure, in particular as part of a closure device of the enclosure.

For example, it may be provided in this case that the interface can be formed by a closure of the nutrient medium.

The advantage of this is that the possibility is thus provided to seal the nutrient medium before its removal.

In a further advantageous embodiment, it may be provided that the controlled environment has a supply line and/or a discharge line for a decontamination agent of the decontamination device, and/or wherein the decontamination device is designed as part of the device.

It is particularly advantageous in this case that different methods of decontamination, for example of the interface, can be used.

For example, it may be provided in this case that the decontamination agent can be supplied to the controlled environment via a fluid flow that is discharged from the cleanroom.

In a further advantageous embodiment, it may be provided that the receptacle is formed in the controlled environment, in particular wherein the receptacle is arranged to open the device outwards.

The receptacle can be horizontally and/or vertically movable, for example.

It is also advantageous that the nutrient medium can be changed independently of a cleanroom process taking place in the cleanroom.

In a further advantageous embodiment, it may be provided that the device comprises a lifting device.

For example, it may be provided in this case that the receptacle can be designed to move vertically.

It is particularly advantageous that, for example, a simple way of removing the nutrient medium is thus realized. In an embodiment for which independent protection is claimed, it may be provided according to the invention that for monitoring for microbiological contamination in a cleanroom, in particular for carrying out a method already mentioned and/or according to one of the claims directed to a device comprising the or a receptacle for presenting one or the nutrient medium, a fluidic connection, in particular from a withdrawal location in the cleanroom, which can be coupled to the nutrient medium, and a means for preferably remote-controlled removal of a closure of the nutrient medium before the fluidic connection is coupled.

The advantage here is that the nutrient medium, which can be provided in an encapsulated seal, can be changed without disturbing the interior of the cleanroom.

Removal can preferably be carried out in an automated or manual manner. For example, this can be carried out fully automatically or manually via a remote control.

A coupling can be, for example, a mechanical connection between the nutrient medium and the fluidic connection or, for example, the opening of a shut-off valve.

A particular advantage of this is that it prevents a contaminated fluid flow from entering the cleanroom.

Another advantage is that the controlled environment can thus be formed by the cleanroom itself.

In a further advantageous embodiment, it may be provided that an elastic membrane is formed outside a flow path of the fluid flow between the isolator and the controlled environment.

The flow path is, for example, the flow area through which a fluid passes during a fluid flow.

It is particularly advantageous that the membrane is fluidically connected to the flow area while a fluid flow is being discharged from the cleanroom into the controlled environment. It is also advantageous that the elasticity of the membrane also allows the fluidic connection to be released as soon as the fluid flow is no longer discharged.

This makes it possible, for example, to remove the seal of the nutrient medium by means of a pivoting movement when the membrane moves vertically.

In a further advantageous embodiment, it may be provided that the membrane is designed as a section of the or an enclosure of the or a controlled environment or the cleanroom.

This means that a movable closure of the controlled environment or the cleanroom, in particular the isolator, can be achieved, especially for changing the nutrient medium.

In a further advantageous embodiment, it may be provided that the device comprises a transfer device, in particular a pivoting device, which is designed with a closure receptacle.

For example, it may be possible to remove the cap of the nutrient medium.

The closure can be an attached lid or a foil, for example.

It is particularly advantageous that the closure can thus be placed outside the flow path of the fluid flow.

For example, it may be provided that the closure receptacle grips the closure of the nutrient medium.

In a further advantageous embodiment, it may be provided that the device comprises the or a fluid line, wherein in particular the interface is formed as part of the fluid line, and/or wherein a flow path of the fluid flow through the fluid line between an opening of the fluid line facing away from the device and the receptacle is less than one meter, preferably less than two or three meters, in particular less than four meters.

It is particularly advantageous that the fluid flow can be discharged as quickly as possible from the cleanroom into the controlled environment via the shortest possible flow path.

In a further advantageous embodiment, it may be provided that the fluid line is closable, in particular when the transfer device is arranged in a remote position.

A particular advantage is that contaminants cannot spread from the controlled volume into the cleanroom.

In a further advantageous embodiment, it may be provided that the or a particle counter is arranged upstream of the nutrient medium in the flow path and/or that the fluid flow is branched off.

For example, it is provided that a flow divider is formed.

For example, it may be provided in this case that the particle counter may be located just before the controlled environment. In addition, it may also be provided, for example, that the particle counter is formed inside the cleanroom.

It is particularly advantageous in this case that a method for monitoring microbiological contamination can be supplemented by quantitative analyses.

Another advantage is that the particle counter can provide information on how many impurities are present, while the nutrient medium can detect what type of impurities have been detected.

In another advantageous embodiment, it may be provided that the fluidic connection can be designed with a device for preventing backflow, in particular a valve.

For example, a valve can be used that has as little contamination surface as possible or as few poorly decontaminable surfaces as possible.

This has the advantage of preventing a contaminated fluid flow from flowing into the cleanroom, which prevents the cleanroom from having to be decontaminated and the cleanroom process from having to be interrupted.

In a further advantageous embodiment, it may be provided that the device comprises a pump, in particular for introducing the fluid flow into the controlled environment.

It is particularly advantageous that the flow direction of the fluid flow can be influenced. This makes it possible, for example, to discharge the fluid flow horizontally and/or vertically from the cleanroom.

In a further advantageous embodiment, it may be provided that the device has at least one sensor for detecting the or a characteristic variable of the fluid flow, and/or wherein the device has a control device for regulating the fluid flow.

It is particularly advantageous that the flow properties of the fluid flow can thus be influenced and/or adapted to the respective conditions.

It is also particularly advantageous that this prevents the flow velocity of the fluid from being too high or too low, which in turn prevents the precise detection of impurities contained in the fluid flow via the nutrient medium.

Alternatively or additionally, one or more of the features directed to a use of a packaged nutrient medium are provided according to the invention for solving said object. In particular, therefore, it is proposed according to the invention for solving the said object in a use, in particular within a cleanroom and/or within a controlled environment, of a packaged nutrient medium, in particular an impactor, for presenting the nutrient medium in a fluid flow, wherein the fluid flow has been taken from a withdrawal location from a cleanroom, in particular an isolator, in particular wherein the impactor has a nozzle plate behind which the nutrient medium is arranged, wherein the nutrient medium, in particular behind the nozzle plate, is covered before use by a closure, in particular a lid, that the closure is gripped with a closure receptacle and removed from the nutrient medium, in particular the nozzle plate, and that a fluidic connection is established from the withdrawal location to the nutrient medium, in particular the nozzle plate.

For example, use can take place within a cleanroom, in particular an impactor, and/or within a controlled environment.

Furthermore, it may be provided, for example, that the nutrient medium, in particular the nozzle plate, is closed after use in the opposite direction by the closure, which is gripped by the closure receptacle, for example.

The impactor is preferably designed to accelerate the fluid flow, in particular via the nozzle plate. The nozzle plate is preferably designed with slots arranged in the circumferential direction, which have such a small opening dimension that impurities can only penetrate through a vacuum. It is particularly advantageous that this allows the fluid flow discharged from the cleanroom to come into contact with the nutrient medium, which in turn makes it possible to monitor for microbiological contamination.

In a further advantageous embodiment, it may be provided that the cleanroom and/or the controlled environment has an opening that can be closed with the closure receptacle, which covers an outer side of the closure.

It is particularly advantageous that the nutrient medium can be sealed with a closure located in the cleanroom or controlled environment for removal. This prevents impurities from entering the nutrient medium after removal. The use of a closure receptacle, with which an opening can be closed, makes it possible to close and remove the nutrient medium closed with the closure through the opening without disturbing the cleanroom or the controlled environment.

Furthermore, it is advantageous that it can be ensured that only the fluid flow that is discharged from the cleanroom contacts the nutrient medium. It is therefore advantageous to prevent a fluid flow that is not discharged from the cleanroom from contacting the nutrient medium and leading to a falsified measurement result.

In a further advantageous embodiment, it may be provided that the impactor is removed from the fluid flow after exposure of the nutrient medium and is supplied to an incubation, in particular wherein the nutrient medium is sealed before removal.

It is particularly advantageous that microbiological contamination can be detected in this way. It is also advantageous that, in the event of contamination, closing the nutrient medium before removal can prevent further surfaces from being contaminated.

In a further advantageous embodiment, it may be provided that the closure receptacle forms a closure of a controlled environment before contacting the closure, in particular on a side facing away from the closure.

It is particularly advantageous that the closure receptacle thus forms an additional boundary to the cleanroom and the probability of contamination of the cleanroom can in turn be reduced. It is also advantageous that contamination of the closure does not necessarily result in contamination of the controlled space.

In another advantageous embodiment, it may be provided that the fluidic connection may comprise a line section.

It is particularly advantageous that the fluid flow can thus be discharged from the cleanroom into the controlled environment.

In another advantageous embodiment, it may be provided that the fluidic connection can be established by means of a coupling that performs a relative movement between the nozzle plate and a line section.

The coupling can, for example, be the fluidic connection between the underside of the line section and the top of the nozzle plate.

It is particularly advantageous that a detachable connection between the nozzle plate and the line section is possible, so that the line sections can be exchanged and/or replaced.

It is also particularly advantageous that this ensures that only the fluid flow that is discharged from the cleanroom comes into contact with the nutrient medium via the coupling.

### BRIEF DESCRIPTION OF THE DRAWINGS

The invention will now be described in more detail with reference to exemplary embodiments, but is not limited to the exemplary embodiments. Further exemplary embodiments are obtained by combining the features of individual or multiple claims with one another and/or with individual or multiple features of the embodiment.

The drawings show in a highly simplified form as follows:

FIG. 1 shows a three-dimensional sectional view of a device for monitoring for microbiological contamination with a nutrient medium carrier in which the nutrient medium is located, a controlled environment, a receptacle, a closure, and a flow path with a fluid line,

FIG. 2 shows a three-dimensional representation of a further device according to the invention with a horizontally and vertically movable closure receptacle and a horizontally movable fluid baffle,

FIG. 3 shows a three-dimensional representation of an alternative embodiment of a device according to FIG. 2,

FIG. 4 shows a three-dimensional representation of a device according to FIG. 3, wherein the receptacle and the fluid baffle are vertically movable,

FIG. 5 shows a two-dimensional sectional view in a front view according to FIG. 4,

FIG. 6 shows a three-dimensional representation of a device with an elastic membrane,

FIG. 7 shows a two-dimensional sectional view as shown in FIG. 6, wherein the receptacle is not in contact and a seal is formed on the nutrient medium,

FIG. 8 shows a two-dimensional sectional view as shown in FIG. 6, wherein the nutrient medium rests against a closure receptacle for removing the closure of the nutrient medium,

FIG. 9 shows a two-dimensional sectional view as shown in FIG. 6, wherein the closure is removed from the nutrient medium and the elastic membrane with a nutrient medium carrier lowered,

FIG. 10 shows a two-dimensional sectional view according to FIG. 6, wherein the closure is positioned within a controlled environment outside a flow path of a fluid flow and the nutrient medium carrier is connected to the fluid line,

FIG. 11 shows a three-dimensional representation of the device according to FIG. 6, additionally with a base plate of the cleanroom and a particle counter positioned in front of the base plate of the cleanroom on a fluid line,

FIG. 12 shows a three-dimensional representation analogous to FIG. 11, wherein the particle counter is positioned inside the cleanroom above the base plate of the cleanroom on the fluid line,

FIG. 13 shows a schematic representation of a setup for continuous monitoring for microbiological contamination with a diverter and two separate devices,

FIG. 14 shows a schematic representation according to FIG. 13 without diverter and with installed valves for fluid flow routing and branching, and

FIG. 15 shows the modified device according to FIG. 6 in the installed state in a cleanroom,

FIG. 16 shows a two-dimensional representation of a further device according to the invention for monitoring for microbiological contamination with a laterally hinged interface.

FIG. 17 shows a two-dimensional representation of a further device according to the invention for monitoring for microbiological contamination with parallel arrangement of the particle counter and the nutrient medium.

### DETAILED DESCRIPTION

A device for monitoring for microbiological contamination, designated with reference sign 1 in its entirety, has a closure receptacle 2 for a closure 3 which can be placed on a nutrient medium carrier 11, wherein the nutrient medium carrier 11 presents a nutrient medium 4 and an impactor 29 which is partially enclosed by an enclosure 13. The enclosure 13 has an opening 24 that can be closed with the closure receptacle 2, which covers an outer side of the closure 3.

The nutrient medium 4 is arranged behind a nozzle plate 25. The impactor 29 is connected to a pump 35, not shown further, via a suction connection 30. The pump thus draws fluid from a fluid line 7 through the nozzle plate 25 past the nutrient medium 4. Any impurities carried along are deposited on the nutrient medium 4 and can then be detected.

A radially widening flow channel with a fluid line 7 and a fluidic connection 27, which comprises a line section 26, is formed above the closure receptacle 2 placed on the nutrient medium 4. The fluid line 7 can extend at a process point within a cleanroom 20, at which a cleanroom process is preferably carried out, and/or partially within the cleanroom 20. It is provided here that the cleanroom 20 is arranged above the fluid line 7.

Thus, a fluid flow 17 can be discharged from the cleanroom 20 from a withdrawal location 28, for example via the aforementioned pump 35, vertically via the fluid line 7 and a flow channel, which defines a flow path of the fluid flow 17, and fed to a controlled environment 5. The fluid flow 17 is thereby accelerated by the nozzle plate 25, which is formed with slots arranged in the circumferential direction with a clear opening dimension.

A controlled environment 5 is bounded by an interface 6, which is first decontaminated by a gas or aerosol, in particular a gas containing hydrogen peroxide, before the interface 6 is made accessible to the fluid flow 17 to be sampled or monitored and after a nutrient medium carrier 11, which does not contain a nutrient medium 4 for decontamination, has been connected to the controlled environment 5.

The gas or aerosol is supplied from the cleanroom 20 and/or from a separate decontamination source, in particular a decontamination device. After decontamination, the nutrient medium carrier 11 is replaced with a nutrient medium carrier 11 containing nutrient medium 4.

The controlled environment 5 thus contains the nutrient medium 4, which is presented by a receptacle 8 on a nutrient medium carrier 11.

To monitor for microbiological contamination, it is provided that the closure 3 of the nutrient medium 4 is first removed via a transfer movement of the closure receptacle 2, which comprises an outer side of the closure 3.

It can therefore be said that the fluid line 7 is only fluidically connected to the nutrient medium 4 after the seal 3 of the nutrient medium 4 has been removed.

The fluid flow 17 can then contact the nutrient medium 4 outside the cleanroom 20 in the decontaminated controlled environment 5.

To remove the nutrient medium 4, this exemplary embodiment provides for a receptacle 8 of the device 1 to be opened outwards via a transfer movement.

When the nutrient medium 4 is replaced, for example, a valve not shown in this figure is activated, which prevents the fluid flow 17 from flowing back.

After contacting the fluid flow 17 discharged from the cleanroom 20 with the nutrient medium 4, it is thus possible to monitor for microbiological contamination.

This can be realized by removing the impactor 29 from the fluid flow 17 after exposing the nutrient medium 4 and feeding it to an incubation after the closure 3 of the nutrient medium 4 has been placed back onto the nutrient medium 4 via a transfer movement of the closure receptacle 2.

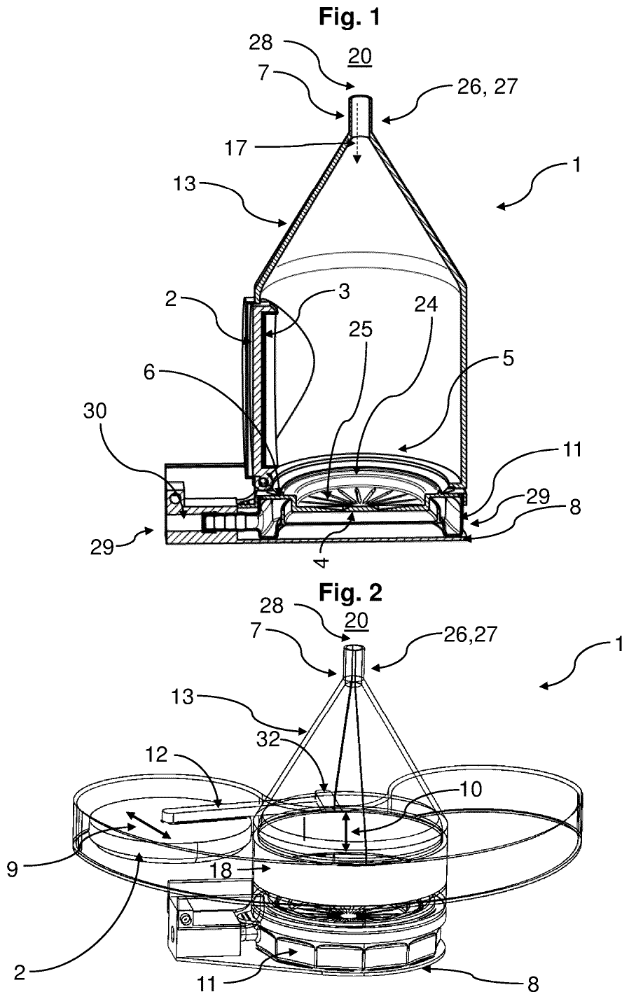

In contrast to FIG. 1, FIG. 2 shows a further device 1 for monitoring for microbiological contamination from the cleanroom 20.

The closure receptacle 2, which is designed to receive the closure 3 of the nutrient medium 4, is attached to the nutrient medium 4 via a lifting device 32 or pivoting device 12, which enables the closure receptacle 2 to perform a lifting movement 10 and/or a pivoting movement 9, whereby the closure 3 of the nutrient medium carrier 11 is removed or placed on the nutrient medium carrier 11.

In addition, this exemplary embodiment shows a fluid baffle 18, which is designed to perform a horizontal pivoting movement 9. The fluid line 7 can thus be closed by pivoting the fluid baffle 18 from the side to a position above the nutrient medium carrier 11 after the closure receptacle 2 with the closure 3 has been pivoted to the side.

Furthermore, it is shown that, in contrast to the preceding exemplary embodiment, it is possible for the closure 3 to be positioned in the controlled environment 5 outside the flow path of the fluid flow 17.

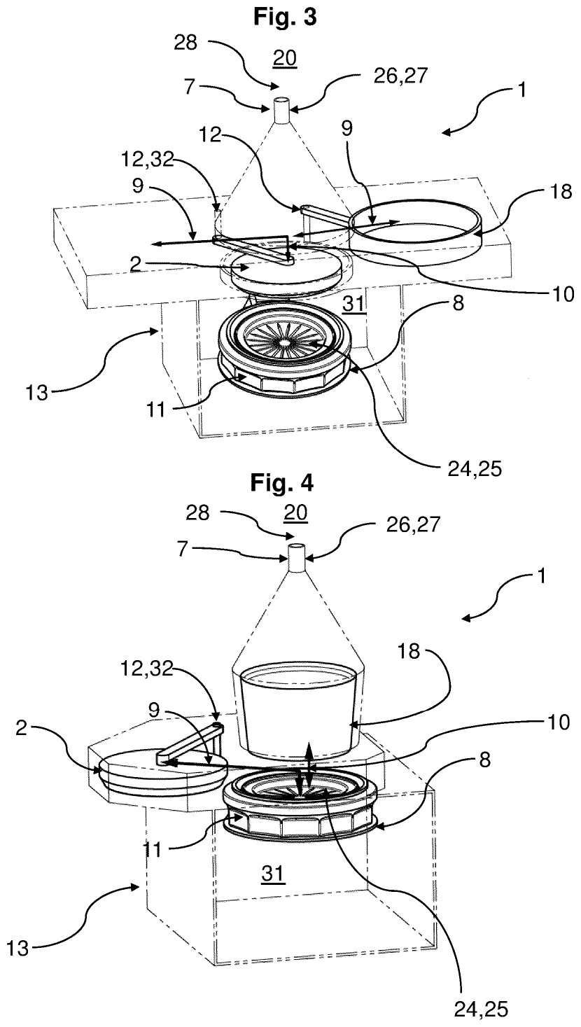

FIG. 3 shows a three-dimensional representation of an alternative version of a device 1 according to FIG. 2. In contrast to FIG. 2, an additional removal space 31 is formed here, which is formed by the enclosure 13.

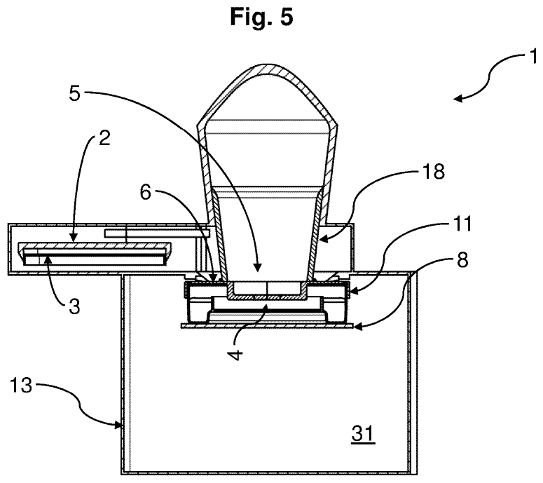

FIG. 4 and FIG. 5 show a further embodiment according to the preceding exemplary embodiments, with the difference that the enclosure 13 does not provide two, but only one placeholder for the closure receptacle 2, which embraces the closure 3. In this exemplary embodiment, the fluid baffle 18 is not pivotable, but is vertically movable in order to produce the connection of the fluid flow 17.

FIG. 5 shows the fluid baffle 18 in the docked state.

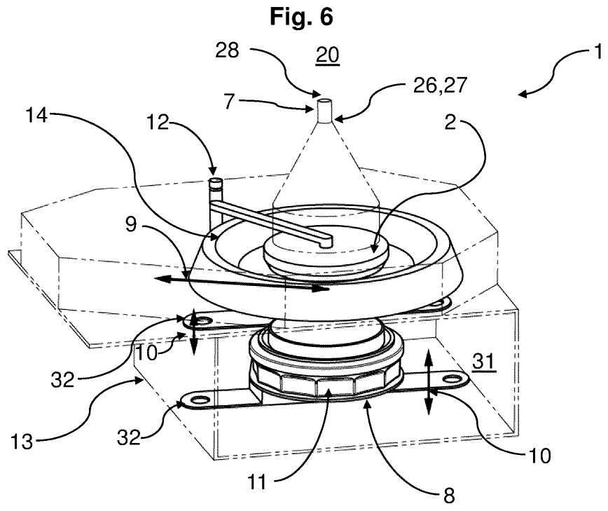

In contrast to the preceding exemplary embodiments, FIG. 6 shows an elastic membrane 14 which is formed outside a flow path of the fluid flow 17 between a removal space 31 and the controlled environment 5 and forms a section of the enclosure 13. The impactor 29 can be removed and supplied via the removal space 31.

Here it is provided that the membrane 4 can be connected or is connected to the nutrient medium 4, in particular to the nutrient medium carrier 11, for removing the closure 3 and/or for coupling the fluid line 7 from the lifting device 32, which realizes a lifting movement 10 of the receptacle 8, and is moved vertically.

FIG. 7 to FIG. 10 show the possible positions due to the vertical movement of the receptacle 8 with the membrane 14.

FIG. 7 shows that the nutrient medium 4, which is positioned in the nutrient medium carrier 11, is not fluidically connected to the membrane 14. In this position, the nutrient medium 4 can be introduced into the controllable environment 5.

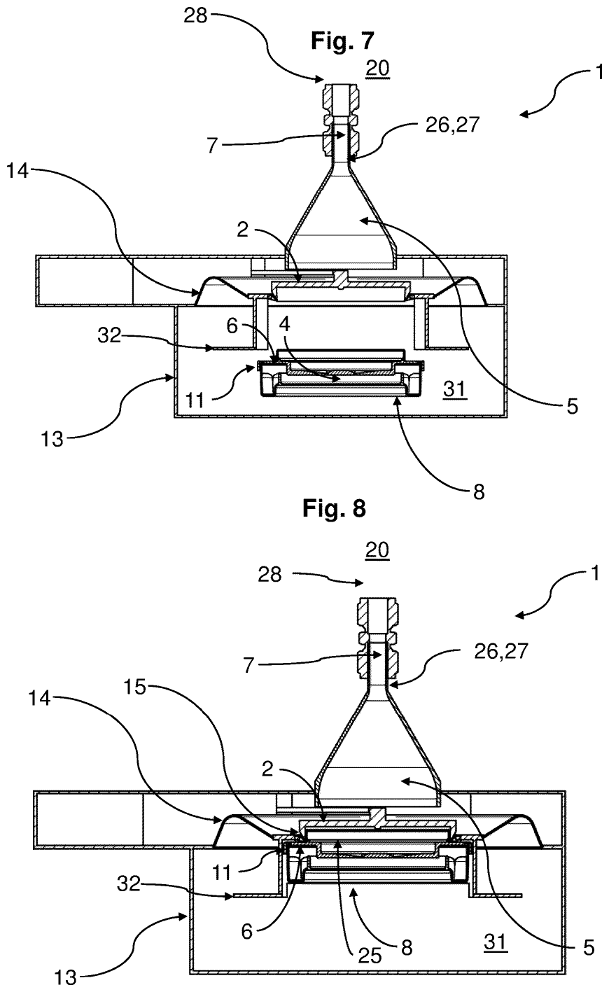

Then, according to FIG. 8, the nutrient medium with the closure 3, in particular the nutrient medium carrier 11, can be lifted to the closure receptacle 2 via the lifting device 32.

Thus, it can be said that a fluidic connection 27, which comprises a line section 26, is established by means of a coupling 15, which performs a relative movement between the nozzle plate 25 and the line section 26.

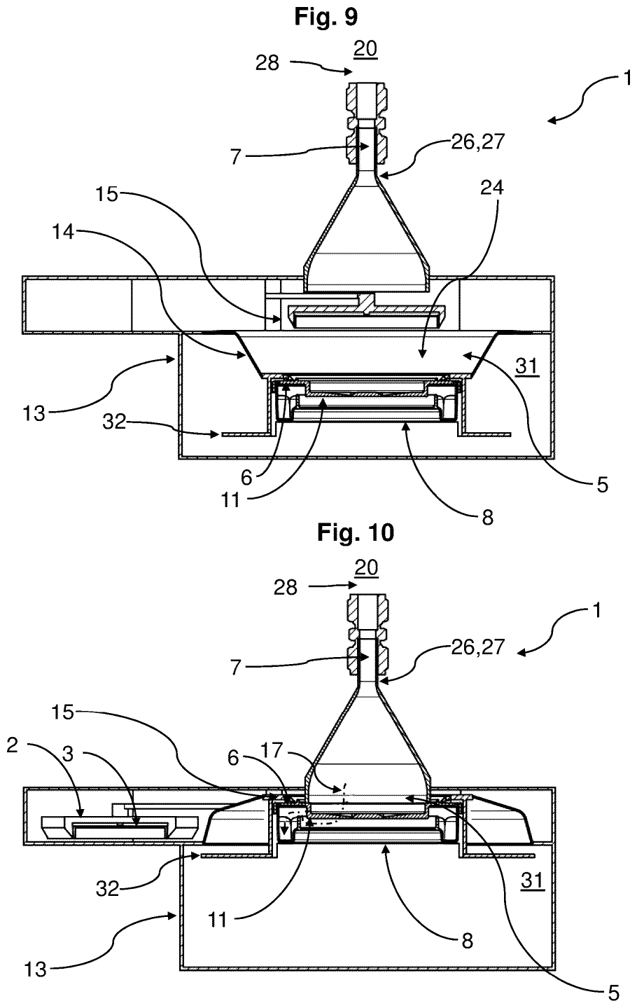

FIG. 9 shows that the nutrient medium carrier 11 is then lowered again, whereby the elastic membrane 14 is also lowered via the connection between the nutrient medium carrier 11 and the elastic membrane 14.

Finally, according to FIG. 10, the closure receptacle 2, which comprises the closure 3 of the nutrient medium 4, can be placed outside the flow path of the fluid flow 17 within the controlled environment 5 by means of a pivoting movement 9 and the nutrient medium 4 can be presented without the closure 3.

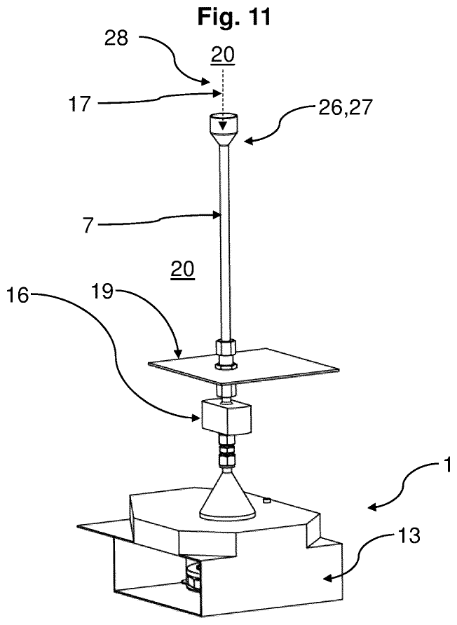

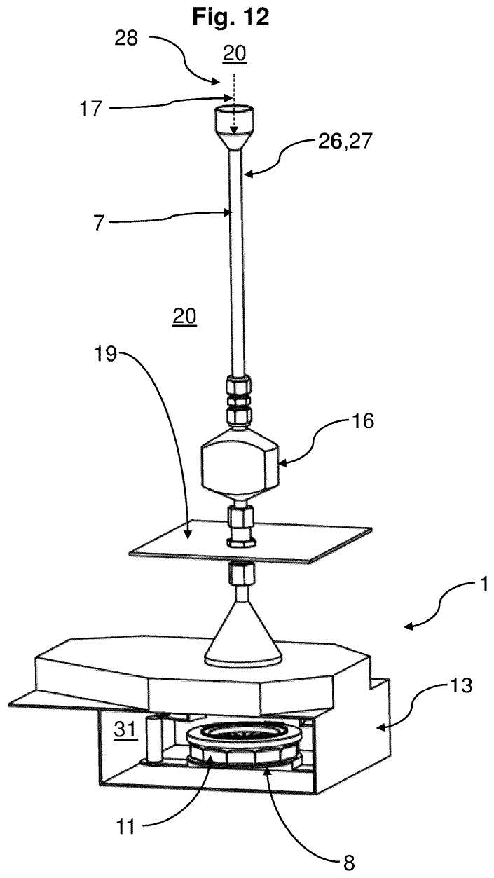

FIGS. 11 and 12 show the entire device 1, with the fluid line 7 extending partially inside the cleanroom 20. In addition, a particle counter 16 is shown, which in FIG. 11 is positioned in front of a base plate 19 of the cleanroom 20, for example an isolator, and in FIG. 12 is positioned inside the cleanroom 20 on the fluid line 7. Thus, the fluid flow 17 discharged from the cleanroom 20 via the fluid line 7 can be passed through the particle counter 16 before contacting the nutrient medium 4.

In other exemplary embodiments, a side wall and/or a ceiling can also be used instead of the base plate 19 to feed through the fluid line 7.

A further variant, not shown in this exemplary embodiment, is to branch off the fluid flow 17, for example outside the cleanroom 20, wherein the branched-off fluid flow 17 is also guided through the particle counter 16.

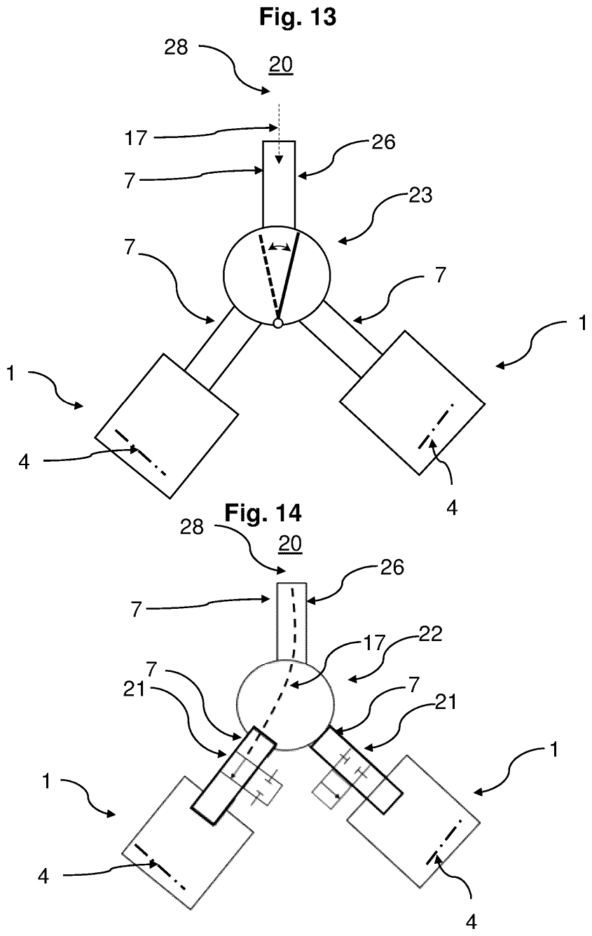

FIG. 13 and FIG. 14 show a basic setup for monitoring for microbiological contamination in a cleanroom 20, wherein, in contrast to the previous exemplary embodiments, two separate devices 1 are used, each of which is designed with a fluid line 7.

The fluid flow 17, which is discharged from the cleanroom 20 for monitoring for microbiological contamination, can thus be directed into two different devices 1 either via an adjustable diverter 23 or via a branch 22 of the fluid line 7.

One of the two devices 1 presents a nutrient medium 4, which is contacted by the fluid flow 17 that is discharged from the cleanroom 20. As soon as the nutrient medium 4, which is presented in one of the devices 1, is to be changed, the fluid flow 17 can be diverted, for example via the diverter 23 or via the branch 22 of the fluid line 7, which is preferably formed with a valve 21, into the other device 1, into which a new nutrient medium 4 is inserted, preferably before the fluid flow 17 is diverted. The branch 22 of the fluid line 7, which is connected to the device 1 in which the nutrient medium 4 is to be changed, is shut off, e.g. via the valve 21, which is in particular in a shut-off position. The valve 21 is located as close as possible to the branch 22 in order to avoid dead spaces, which means that continuous monitoring for microbiological contamination can be realized.

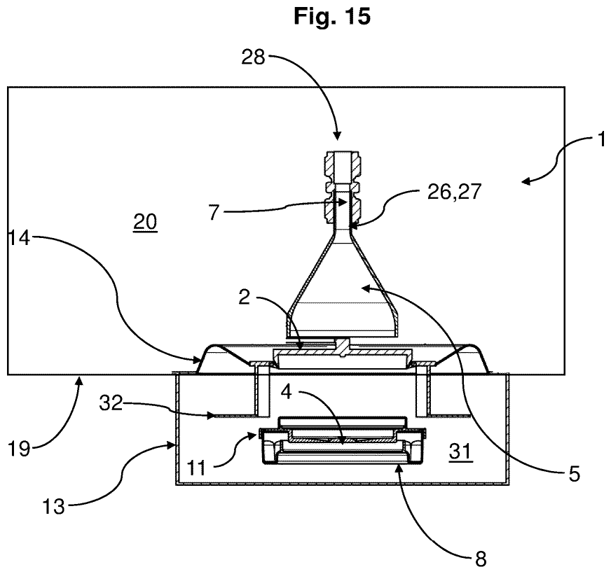

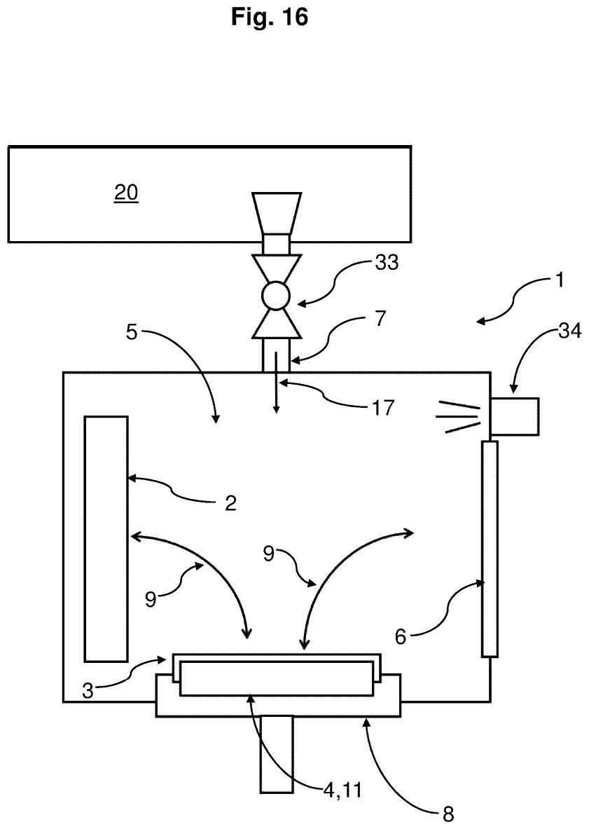

FIG. 15 shows the modified device 1 according to FIG. 6 in an installed state in a cleanroom 20. It can be seen that the controlled environment 5 is formed by the cleanroom 20 and that the membrane 14 is formed as a section of the cleanroom 20. It can also be seen that the controlled environment 5 can be separated from the cleanroom 20, except for the fluid line 7.

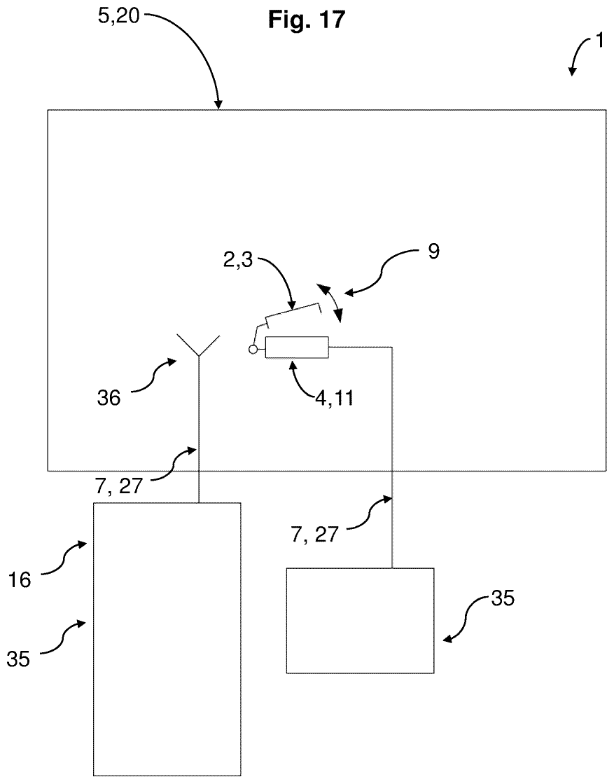

FIG. 16 shows a further device 1 according to the invention having an interface 6 which laterally delimits the controlled environment 5 and can be hinged via a pivoting movement 9 and which is decontaminated at least on the inside before the interface 6 is made accessible for the fluid flow 17 to be monitored. In order to counteract a return flow of the fluid flow 17 discharged from the cleanroom 20, preferably isolator, a backflow prevention device, which in this exemplary embodiment is designed as a ball valve 33 between the controlled environment 5 and the cleanroom 20, is attached to the fluid line 7. In an exemplary embodiment not shown, another backflow prevention device, for example a membrane valve or another shut-off device known to the person skilled in the art, is fitted instead of the ball valve 33.

In this exemplary embodiment, the decontamination agent required for decontamination is supplied to the interface 6 and the controlled environment 5 via a nozzle 34.

In addition, similar to the preceding exemplary embodiments, it can be seen that the closure 3 is removed from the nutrient medium carrier 11 presenting the nutrient medium 4 via a pivoting movement 9 of the closure receptacle 2 or is placed on the nutrient medium carrier 11 to close the nutrient medium 4.

FIG. 17 shows a further device for monitoring for microbiological contamination 1. It shows a fluid line 7 or fluidic connection 27 with a particle counter 16, which takes fluid samples, in this case gas samples, at a sampling point 36 within the controlled environment 5, which in this exemplary embodiment is formed by the cleanroom 20 itself. Here, a controlled fluid flow 17 is drawn in via a pump 35 arranged outside the cleanroom 20 (on the left in the figure) and thus fed to the particle counter 16.

In addition, the nutrient medium carrier 11 with the nutrient medium 4 presented therein is also located within the controlled environment 5 or cleanroom 20 parallel to the particle counter 16. The nutrient medium carrier 11 is connected to a further pump 35 arranged outside the cleanroom 20 via a further fluid line 7 or fluidic connection 27, via which the fluid flow 17 is drawn in, which in turn enables defined contact between the fluid flow 17 of the cleanroom 20 and the nutrient medium 4. It is provided in this case that continuous monitoring for microbiological contamination is initially carried out via the particle counter 16, wherein the nutrient medium 4 is initially sealed by the closure 3 and is therefore not presented. The pump 35 (in the right-hand image), which is connected to the nutrient medium carrier 11 via the fluid line 7, does not initially draw in any fluid flow 17.

As soon as objects are detected by the particle counter 16, the closure 3 of the nutrient medium 4 is automatically removed from the closure receptacle 2 via a pivoting movement 9 and the nutrient medium 4 is presented. The (right) pump 35, which is connected to the nutrient medium carrier 11 via the fluid line 7, sucks in the fluid flow 17, which in turn contacts the presented nutrient medium 4.

It can therefore be said that the nutrient medium 4 is only presented in the fluid flow 17 when impurities have been detected by the particle counter 16. After presentation, the nutrient medium 4 can be examined in a manner known per se to determine whether the contamination is capable of multiplying. In an exemplary embodiment not shown, it may further be provided that the nutrient medium 4 is located outside the cleanroom 20 within a controlled environment 5. According to FIG. 16, the fluid line 7 or fluidic connection 27 connects the cleanroom 20 with a controlled environment 5, for example as in the preceding embodiments, and is additionally designed with a switchable valve 21. As soon as the particle counter 16 has detected microbiological contaminations, the valve 21 is switched to an open position, whereby the nutrient medium 4 is again automatically presented and contacted by the fluid flow 17.

In a further exemplary embodiment, the nutrient medium 4 and the particle counter 16 are connected to a common pump 35. The nutrient medium 4 can thus be arranged in a switchable bypass to the fluid flow of the particle counter 16.

In a further exemplary embodiment, the nutrient medium 4 can also be provided with an upstream fluid line 7, in particular one that can be disconnected (see, for example, FIG. 12).

According to the invention, it is thus proposed for monitoring for microbiological contamination that a fluid flow 17 is discharged via a fluid line 7 from a cleanroom 20 into a controlled environment 5, wherein a nutrient medium 4 is presented in the controlled environment 5 and with which the fluid flow 17 is contacted and subsequently removed from the controlled environment 5 in such a way that a cleanroom process does not have to be interrupted.

### LIST OF REFERENCE SIGNS

1 Device for monitoring for microbiological contamination
2 Closure receptacle
3 Closure
4 Nutrient medium
5 Controlled environment
6 Interface
7 Fluid line
8 Receptacle
9 Pivoting movement
10 Lifting movement
11 Nutrient medium carrier
12 Pivoting device
13 Enclosure
14 Elastic membrane
15 Coupling
16 Particle counter
17 Fluid flow
18 Fluid baffle
19 Base plate of the cleanroom
20 Cleanroom
21 Valve
22 Branch
23 Diverter
24 Opening
25 Nozzle plate
26 Line section
27 Fluidic connection
28 Withdrawal location
29 Impactor
30 Suction connection
31 Withdrawal room
32 Lifting device
33 Ball valve
34 Nozzle
35 Pump
36 Sampling point

1. A method for monitoring for microbiological contamination in a cleanroom (20), the method comprising:
discharging a fluid flow (17) from the cleanroom (20);
contacting the fluid flow (17) with a nutrient medium (4) in a controlled environment (5) arranged outside the cleanroom (20), the controlled environment (5) is at least one of connected by a fluid line (7) or separable from the cleanroom (20); and
using the nutrient medium (4) for monitoring for microbiological contamination.

2. The method according to claim 1, further comprising decontaminating an interface (6) delimiting the controlled environment (5) at least on an inside before the interface (6) is made accessible for the fluid flow (17) to be monitored.

3. The method according to claim 2, further comprising forming a branch (22) or a diverter (23) on the fluid line (7).

4. The method according to claim 2, further comprising supplying a decontamination agent for decontaminating at least one of the interface (6) or the controlled environment (5).

5. The method according to claim 1, further comprising at least one of a) the discharging of the fluid flow (17) from the cleanroom (20) being at a process point within the cleanroom (20), at which a cleanroom process is carried out, or b) supplying the fluid flow (17) to the nutrient medium (4) in the fluid line (7) which runs at least partially within the cleanroom (20).

6. The method according to claim 1, further comprising a) passing the fluid flow (17 through a particle counter (16) for detecting at least one of a particle count or particle size, and subsequently passing the same fluid flow (17) to the nutrient medium (4), or b) branching a partial volume of the fluid flow (17) to a particle counter (16) for detecting at least one of a particle count or particle size.

7. The method according to claim 1, wherein the nutrient medium (4) is introduced into the controlled environment (5) from the outside and positioned at a receptacle (8).

8. A method for monitoring for microbiological contamination in a cleanroom (20), the method comprising:
discharging a fluid flow (17) from the cleanroom (20);
contacting the fluid flow (17) with a nutrient medium (4);
using the nutrient medium (4) for monitoring for microbiological contamination; and
connecting a fluid line (7), which brings the fluid flow (17) to the nutrient medium (4), to the nutrient medium (4) after removing a closure (3) of the nutrient medium (4).

9. The method according to claim 8, further comprising removing the closure (3) via a transfer movement including at least one of a horizontal pivoting movement (9) or a lifting movement (10).

10. The method according to claim 9, further comprising carrying out a particle detection in the fluid flow (17) or a further fluid flow (17) and presenting the nutrient medium (4) in the fluid flow (17) when the particle detection has detected at least one of a reproducible or living object, by at least one of diverting the fluid flow (17), opening a fluid line (7) conducting the fluid flow (17), or starting the fluid flow (17).

11. A device (1) for monitoring for microbiological contamination in a cleanroom (20), the device comprising:
an enclosure (13) which encloses a controlled environment (5);
a receptacle (8) for presenting a nutrient medium (4) in the controlled environment (5);
a fluidic connection (27) for connection to the cleanroom (20) and an interface (6), which delimits the controlled environment (5), for providing the nutrient medium (4).

12. The device (1) according to claim 11, wherein the fluidic connection (27) comprises a branch (22) or a diverter (23) which is designed to divert a fluid flow (17).

13. The device (1) according to claim 11, further comprising a decontamination device for decontaminating at least the interface (6).

14. The device (1) according to claim 13, wherein the interface (6) is part of a closure device of the enclosure (13).

15. The device (1) for monitoring for microbial growth in a cleanroom as claimed in claim 11, further comprising the fluidic connection (27) at a withdrawal location (28) in the cleanroom (20), which is couplable to the nutrient medium (4), and a remote-controlled closure removal device for removal of a closure (3) of the nutrient medium (4) before the fluidic connection (27) is coupled.

16. The device (1) according to claim 15, further comprising an elastic membrane (14) formed outside a flow path of the fluid flow (17) between an isolator and the controlled environment (5).

17. The device (1) according to claim 15, further comprising a transfer device that comprises a pivoting device (12), which is formed with a closure receptacle (2).

18. The device (1) according to claim 17, wherein the interface (6) is formed as part of the fluid line (7), and a flow path of the fluid flow (17) through the fluid line (7) between an opening (24) of the fluid line (7) facing away from the device (1) and the receptacle (8) is less than four meters.

19. The device (1) according to claim 11, further comprising a particle counter (16) arranged upstream of the nutrient medium (4) in the flow path.

20. The device (1) according to claim 11, further comprising at least one of: a) a sensor for detecting a characteristic variable of the fluid flow (17), or b) a control device for regulating the fluid flow (17).

21. A method of operating at least one of a cleanroom (20) or a controlled environment (5), the method comprising:
using an impactor (29) for presenting a packaged nutrient medium (4) in a fluid flow (17);
taking the fluid flow (17) from a withdrawal location (28) from the cleanroom (20) or controlled environment (5);
the impactor (29) including a nozzle plate (25) behind which the nutrient medium (4) is arranged, with the nutrient medium (4) being covered before use by a closure (3);
gripping the closure (3) with a closure receptacle (2) and removing the closure (3) from the nutrient medium (4); and
establishing a fluidic connection (27) from the withdrawal location (28) to the nutrient medium (4) at the nozzle plate (25).

22. The method according to claim 21, wherein the at least one of the cleanroom (20) or the controlled environment (5) has an opening (24) that is closeable with the closure receptacle (2), which covers an outer side of the closure (3).

23. The method according to claim 21, further comprising removing the impactor (29) from the fluid flow (17) after exposure of the nutrient medium (4) and supplying the impactor (29) to an incubation.

24. The method according to claim 21, wherein the closure receptacle (2) forms a closure of the controlled environment (5) before contacting the closure (3).
